(19) United States
(12) Patent Application Publication
(10) Pub. No.: US 2024/0293179 A1
(43) Pub. Date: Sep. 5, 2024

(54) MICROWAVE ABLATION SYSTEM

(71) Applicant: Covidien LP, Mansfield, MA (US)

(72) Inventor: William J. Dickhans, Longmont, CO (US)

(21) Appl. No.: 18/655,445

(22) Filed: May 6, 2024

Related U.S. Application Data
(63) Continuation of application No. 16/853,925, filed on Apr. 21, 2020, now Pat. No. 11,974,805.
Application No. 16/853,925 is a continuation of application No. 14/828,682, filed on Aug. 18, 2015, now Pat. No. 10,624,697.
(60) Provisional application No. 62/041,773, filed on Aug. 26, 2014.

(51) Int. Cl. A61B 18/18; A61B 18/00; A61B 34/10; A61B 34/20; A61B 90/00

(52) U.S. Cl. CPC A61B 18/1815; A61B 90/39; A61B 2018/00017; A61B 2018/00023; A61B 2018/00029; A61B 2018/00511; A61B 2018/00541; A61B 2018/00577; A61B 2018/1861; A61B 2034/107; A61B 2034/2051

(57) ABSTRACT

A microwave ablation catheter assembly is provided. A coaxial cable configured to connect to a microwave energy source at its proximal end and at its distal end to a distal radiating section. The coaxial cable includes inner and outer conductors and a dielectric positioned therebetween. An extended working channel is configured to receive the coaxial cable for positioning the coaxial cable adjacent target tissue. At least a portion of an inner surface of the extended working channel is electrically conductive. The electrically conductive inner surface of the extended working channel may function as a balun to maintain a balanced signal between the inner and outer conductors of the coaxial cable when the distal radiating section of the coaxial cable is energized.

### CROSS-REFERENCE TO RELATED APPLICATIONS

This application is a continuation of U.S. patent application Ser. No. 16/853,925, filed on Apr. 21, 2020, now U.S. Pat. No. 11,974,805, which is a continuation of U.S. patent application Ser. No. 14/828,682, filed on Aug. 18, 2015, now U.S. Pat. No. 10,624,697, which claims the benefit of the filing date of provisional U.S. Patent Application No. 62/041,773, filed on Aug. 26, 2014.

### BACKGROUND

The present disclosure relates to a microwave ablation catheter assembly. More particularly, the present disclosure relates to a microwave ablation catheter assembly including an extended working channel having an electrically conductive inner surface that functions as an electromagnetic shield or a balun for a microwave ablation catheter of the microwave ablation catheter assembly.

Microwave ablation involves the application of high-frequency electromagnetic waves for treating various maladies including tumors in of organs like the liver, brain, heart, lung and kidney. It is known that tumor cells denature at elevated temperatures that are slightly lower than temperatures injurious to surrounding healthy cells. Therefore, known treatment methods, such as hyperthermia therapy, heat tumor cells to temperatures above 41° C., while maintaining adjacent healthy cells at lower temperatures to avoid irreversible cell damage. Such methods may involve applying electromagnetic radiation, or microwave energy, to heat tissue and include ablation and coagulation of tissue. In particular, microwave energy is used to ablate tissue to denature or kill the cancerous cells.

Conventional microwave ablation systems typically include one or more microwave ablation catheters coupled to a microwave energy source via a feedline, typically in the form of a coaxial cable. The microwave ablation catheters are placed adjacent target tissue and microwave energy is applied to the microwave ablation catheters causing localized heating of target tissue. The microwave ablation catheter is typically relatively thin and flexible to allow a user to navigate the microwave ablation catheter through a luminal network of one of the aforementioned organs, e.g., a lung.

When treating malignancies of the lung, microwave ablation systems are often used in conjunction with an electromagnetic navigation (EMN) system. One such system is described in U.S. Pat. No. 6,188,355 and published PCT Application Nos. WO 00/10456 and WO 01/67035, the entire contents of which are hereby incorporated by reference. An EMN system typically includes a bronchoscope, a catheter assembly containing a location sensor at its steerable distal tip, an extended working channel that extends beyond the reach of the bronchoscope and becomes a pathway to the target site for subsequent diagnostic tools, and a computer system which provides the physician, or user, with navigational views of the lung. Once the bronchoscope is inserted into a patient's lungs, the catheter assembly with the extended working channel is inserted into the bronchoscope. Using the navigation system and the steerable distal tip, the catheter assembly and extended working channel is navigated to a target location. The catheter assembly is then removed, leaving the extended working channel in place. The microwave ablation catheter can then be inserted into the extended working channel and directed to the target location.

As previously mentioned, the microwave ablation catheter is coupled to the microwave energy source via a feedline, often in the form of an unbalanced coaxial cable. As a result of the coaxial cable being unbalanced, there is often a loss of microwave energy along the coaxial cable. Additionally, substantial heating of the coaxial cable can occur during delivery of microwave energy. In order to help minimize the loss of energy as a result of an unbalanced coaxial cable, microwave ablation catheters that utilize a feedline in the form of a coaxial cable may include a balun or choke. The balun or choke helps to balance the coaxial cable of the microwave ablation catheter when microwave energy is transmitted to the radiating section of the microwave ablation catheter to ablate tissue and substantially impede the current flowing down the outer conductor which might lead to undesired heating of tissue along the length of the ablation catheter.

While the aforementioned microwave ablation catheters are suitable for their intended purposes, the balun on the microwave ablation catheter is added structure that increases the size of the microwave ablation catheter, which, in turn, may decrease the flexibility of the microwave ablation catheter.

### SUMMARY

As can be appreciated, an extended working channel having an electrically conductive inner surface that functions as an electromagnetic shield and/or a balun for a microwave ablation catheter of a microwave ablation system may prove useful in the surgical arena.

Aspects of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

As defined herein braided means made by intertwining three or more strands, and while described as a braid, the actual construction is not so limited and may include other formations as would be understood by those of ordinary skill in the art.

An aspect of the present disclosure provides a microwave ablation catheter assembly comprising an ablation catheter and an extended working channel. The ablation catheter includes a coaxial cable having a proximal portion and a distal portion and a radiator disposed at the distal portion of the coaxial cable. The coaxial cable further includes an inner conductor and an outer conductor. The proximal portion of the coaxial cable is capable of being operatively connected to a microwave energy source. The extended working channel is configured to receive the ablation catheter for positioning the radiator adjacent target tissue. The extended working channel includes an electrically conductive inner surface, wherein, upon application of microwave energy to the microwave ablation catheter assembly, energy conducted along the outer conductor of the coaxial cable is captured within the conductive inner surface of the extended working channel and prevented from affecting tissue adjacent the extended working channel.

The microwave ablation catheter assembly, including the ablation catheter and extended working channel, may me placed within a patient using a location sensing system. The extended working channel may also include a slot at its proximal end configured to releasably engage with a corresponding mechanical interface positioned on the ablation catheter. The mechanical interface may also be configured to be moveable with the slot to lock the distal radiating section of the coaxial cable into at least one of a plurality of positions defined within the slot. Indicia may also be provided along the slot and may be configured to represent quarter-wavelength increments. The extended working channel may further comprise an insulator separating the electrically conductive inner surface from tissue adjacent the extended working channel.

The ablation catheter of the microwave ablation catheter assembly may further include one or more cooling catheters surrounding the coaxial cable and radiator to provide a pathway for a cooling medium, such as a gas or liquid. The extended working channel may also provide for an open or closed pathway for a cooling medium to either circulate within or pass through the extended working channel.

An aspect of the instant disclosure provides a method of electrosurgically treating target tissue. An extended working channel, having at least a portion of an electrically conductive inner surface, is positioned adjacent target tissue. Thereafter, an ablation catheter, having an outer conductor, is inserted through the extended working channel such that a radiator of the ablation catheter extends beyond the distal end of the extended working channel. Energy is then applied to the ablation catheter such that the electrosurgical energy radiates from the radiator to electrosurgically treat the target tissue. Upon application of energy to the ablation catheter, any energy conducted along an outer conductor of the ablation catheter is captured within the electrically conductive inner surface of the extended working channel, preventing the energy from affecting tissue adjacent the extended working channel.

The method of electrosurgically treating target tissue may further include engaging a mechanical interface of the ablation catheter with at least one mechanical interface defined along a slot provided on the extended working channel to lock the radiator of the ablation catheter in a position that is distal of the distal end of the extended working channel. The slot may be provided at a proximal end of the extended working channel and may be configured to releasably couple to a mechanical interface positioned on the ablation catheter. Indicia may further be provided along the slot which may represent quarter-wavelength increments. The method may further include moving the mechanical interface of the ablation catheter distally to lock the ablation catheter into at least one other position within the slot to adjust a signal balance between inner and outer conductors of the ablation catheter. The electrically conductive inner surface of the extended working channel may further include a braided configuration.

An aspect of the present disclosure provides a microwave ablation system. The microwave ablation system comprises a microwave energy source, an ablation catheter, and an extended working channel. The ablation catheter includes a coaxial cable, having a proximal portion and a distal portion, and a radiator disposed at the distal portion of the coaxial cable. The coaxial cable includes an inner conductor and an outer conductor, and the proximal portion of the coaxial cable is operatively connected to the microwave energy source. The extended working channel is configured to receive the ablation catheter for positioning the radiator adjacent target tissue. The extended working channel includes an electrically conductive inner surface, wherein, upon application of microwave energy to the ablation catheter, energy conducted along the outer conductor of the coaxial cable is captured within the conductive inner surface of the extended working channel and prevented from affecting tissue adjacent the extended working channel.

### BRIEF DESCRIPTION OF THE DRAWINGS

Various embodiments of the present disclosure are described herein with references to the drawings, wherein:

FIG. 1 is a schematic illustration of an EMN system configured for use with a microwave ablation catheter in accordance with an illustrative embodiment of the present disclosure;

FIG. 2 is a perspective view of a microwave ablation catheter in accordance with an embodiment of the instant disclosure;

FIG. 3A is a perspective view of the distal and proximal portions of an extended working channel of a catheter guide assembly shown in FIG. 1;

FIG. 3B is a transverse, cross-sectional view of one embodiment of the extended working channel shown in FIG. 3A; and

FIG. 4 is a perspective view of the microwave ablation catheter positioned within the extended working channel and with a distal radiating section positioned within target tissue.

FIG. 5 is a perspective view of the microwave ablation catheter positioned within a metal hypo tube in accordance with the present disclosure.

### DETAILED DESCRIPTION

The present disclosure is directed to a microwave ablation catheter assembly and a method for placement of a microwave ablation antenna within a luminal structure such as the pathways of the bronchi in the lungs. Embodiments of the present disclosure include an un-choked microwave ablation catheter, or microwave ablation catheter without a balun. Further embodiments are directed to a microwave ablation catheter with a modified balun or choke. Still further embodiments of the present disclosure are directed to an improved microwave ablation catheter assembly having increased flexibility and a reduced number of components while providing adequate therapeutic results.

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

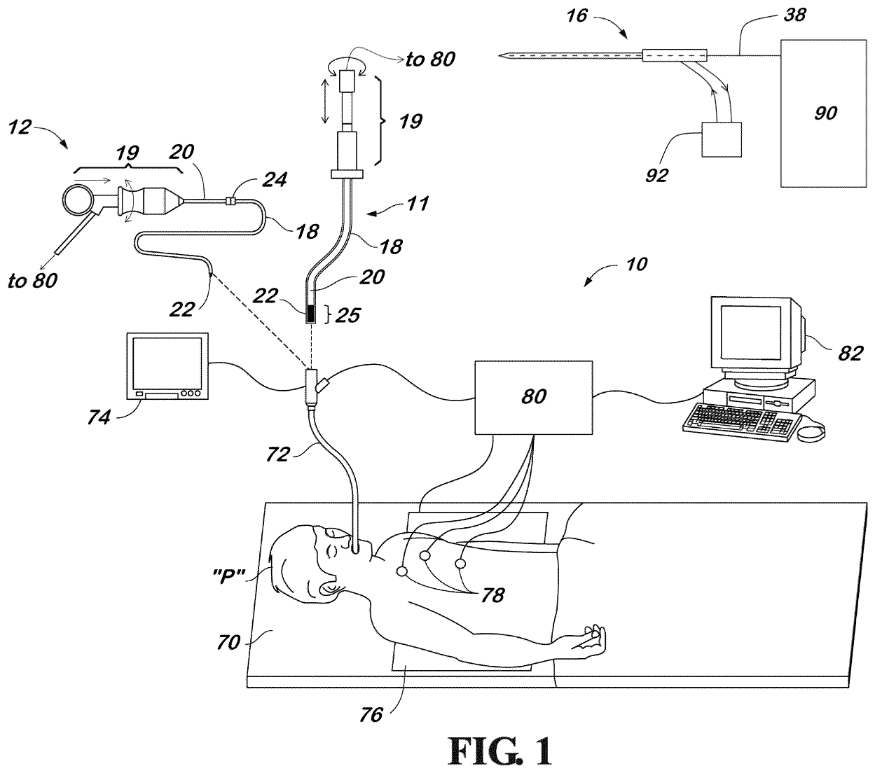

FIG. 1 is an illustration of an EMN system 10 in accordance with the present disclosure. One such EMN system 10 is the ELECTROMAGNETIC NAVIGATION BRONCHOSCOPY® system currently sold by Covidien LP. Typically the EMN system 10 includes a bronchoscope 72, one or more of two different types of catheter guide assemblies 11 and 12, monitoring equipment 74, an electromagnetic field generator 76, a tracking module 80, and a computer system 82. FIG. 1 shows a patient “P” lying on an operating table 70 including an electromagnetic field generator 76. Placed on the patient “P” are a number of sensors 78, whose position in the magnetic field generated by the electromagnetic field generator 76 can be determined by the tracking module 80.

Each of the catheter guide assemblies 11, 12 includes an extended working channel 18 that is configured to receive a locatable guide catheter 20 which includes a sensor 22. The locatable guide catheter 20 is electrically connected to the EMN system 10, and particularly, the tracking module 80 enables navigation and tracking of the sensor 22 within a luminal network, such as the lungs of a the patient “P”, to arrive at a designated target. As will be described in greater detail below, the extended working channel 18 is configured to receive instruments including the locatable guide catheter 20 and sensor 22, biopsy tools and microwave ablation catheter 16, as well as others without departing from the scope of the present disclosure.

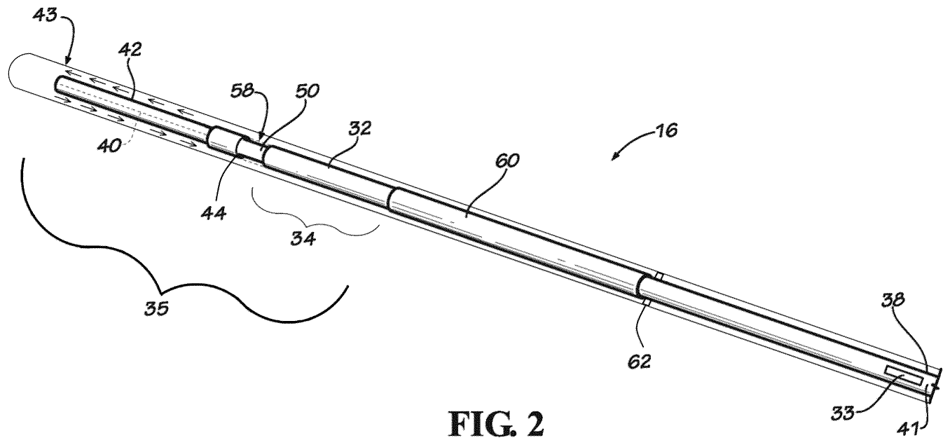

FIGS. 1 and 2 depict an ablation catheter 16 in accordance with one embodiment of the present disclosure. The ablation catheter 16 includes a coaxial cable 38. Coaxial cable 38 includes a proximal end 41 that couples to a microwave energy source 90 (FIG. 1). A cooling source 92 connects to the ablation catheter 16 to circulate cooling fluid as will be described in greater detail below. As shown in greater detail in FIG. 2, a distal radiating section 42 is provided at a distal end 44 of the coaxial cable 38 and is configured to receive the inner conductor 40. The distal radiating section 42 may be formed from any suitable material. For example, in embodiments, the distal radiating section 42 may be formed from ceramic or metal, e.g., copper, gold, silver, etc. The distal radiating section 42 may include any suitable configuration including but not limited to a blunt configuration, flat configuration, hemispherical configuration, pointed configuration, bar-bell configuration, tissue piercing configuration, etc. The distal radiating section 42 may couple to the distal end 44 of the coaxial cable via soldering, ultrasonic welding, adhesive, or the like. In one embodiment the distal radiating section 42 is sealed to the inner conductor 40 to prevent fluid from contacting the inner conductor 40.

Proximate the distal radiating section 42 is a feed gap 58, which is formed by removing a portion of the outer conductor 32 of the coaxial cable 38, to expose a dielectric 50. Proximate the feed gap 58 is the proximal radiating section 34, which is essentially just a portion of the outer conductor 32 of the coaxial cable 38. The proximal radiating section 34, the feedgap 58, and the distal radiating section 42 are located and dimensioned to achieve a specific radiation pattern for the ablation catheter 16 and in combination are collectively known as the radiator 35 of the ablation catheter 16. As will be explained in greater detail below, the extension of the radiator 35 out of the extended working channel 18 enables the ablation of tissue, but the length of that extension can be varied as desired to adjust the shape and size of the ablation zone.

The outer conductor 32 is typically formed of a braided electrically conducting material and extends along the dielectric 50, which is positioned between the inner and outer conductors 40 and 32, respectively. One advantage of a braided configuration of the outer conductor 32 is that it provides the ablation catheter 16 with the flexibility to move within the relatively narrow luminal structures such as the airways of the lungs of a patient. Additionally, through the use of flat wire braiding and follow on braid compression with an appropriately sized die, the cross sectional dimension of the braided conductor may be minimized significantly in comparison to other conductive structures, while maintaining an acceptable electrical performance. The ablation catheter 16 may include one or more cooling catheters 43 surrounding the coaxial cable 38 and radiator 35, these cooling catheters 43 enable the passage of cooling medium over the coaxial cable 38 and the radiator 35. The cooling catheters provide pathways for cooling liquid or gas to reach the distal radiating section and remove heat generated by the application of energy. Cooling the radiator 35 and the coaxial cable 38 helps ensure that the ablation process is undertaken by the radiating waves of electromagnetic energy heating the tissue and not by localized heating of the coaxial cable 38 or distal radiating section 42. Though shown in FIG. 2 with a single cooling catheter 43, those of skill in the art will appreciate that additional co-luminal catheters may be employed to enable bi-directional cooling medium flow with cooling medium passing the coaxial cable 38 and a first cooling catheter in a distal direction, reaching an area proximate the distal end of the ablation catheter and then returning in a proximate direction between the first and a second cooling catheter. Further, as will be appreciated by those of skill in the art, the cooling catheters 43 are not entirely necessary and ablation catheter 16 may be utilized in an uncooled or open cooled system as will be described in greater detail below.

The flexibility of the ablation catheter 16 can be altered to accommodate a specific surgical procedure, a specific luminal structure, specific target tissue, a clinician's preference, etc. For example, in an embodiment, it may prove advantageous to have an ablation catheter 16 that is very flexible for movement through the relatively narrow airway of the lungs of a patient. Alternatively, it may prove advantageous to have an ablation catheter 16 include portions which are less flexible, e.g., where the ablation catheter 16 is needed to pierce or puncture target tissue.

One of the effects of ablation catheters is an energy loss that occurs as energy intended for radiation into the tissue via the radiator 35 is reflected and/or travels proximally along the outer conductor 32 of the coaxial cable 38, resulting in a less efficient system. In some instances this energy can cause heating along the length of the catheter, and affect the tissue proximate the outer conductor 32. As noted above, one methodology for preventing the traversal of this energy along the outer conductor is to employ a balun or choke which effectively causes the energy to be reflected back towards the distal radiating section 42, and provide useful energy for the ablation process, rather than be an energy loss in the system. Methods of forming a balun for a flexible ablation catheter are described in U.S. patent application Ser. No. 13/834,581, filed on Mar. 15, 2013 by Brannan et al. entitled “Microwave Energy-Delivery Device and System,” the entire contents of which are hereby incorporated by reference.

However, to improve the flexibility of the ablation catheter 16, one embodiment of the present disclosure does not employ a balun or choke. Instead, the ablation catheter 16, as shown in FIG. 2, relies on the construction of the components of the catheter guide assemblies 11 and 12, and particularly the extended working channel 18 to control the emission of microwave energy from the ablation catheter 16, substantially contain the energy traveling down the outer conductor 32, and prevent that energy from affecting tissue.

To further clarify how the construction of the catheter guide assemblies 11, 12 affect the emission of microwave energy, reference is made to FIG. 1 which depicts two types of catheter guide assemblies 11, 12. Both catheter guide assemblies 11, 12 are usable with the EMN system 10 and share a number of common components. Each catheter guide assembly 11, 12 includes a handle 19, which is connected to an extended working channel 18. The extended working channel 18 is sized for placement into the working channel of a bronchoscope 72. In operation, a locatable guide 20, including an electromagnetic sensor 22, is inserted into the extended working channel 18 and locked into position such that the sensor 22 extends a desired distance beyond the distal tip 25 of the extended working channel 18. The location of the sensor 22, and thus the distal end of the extended working channel 18, within an electromagnetic field generated by the electromagnetic field generator 76 can be derived by the tracking module 80, and the computer system 82. Catheter guide assemblies 11, 12 have different operating mechanisms, but each contain a handle 19 that can be manipulated by rotation and compression to steer the distal tip 25 of the extended working channel 18 and/or the sensor 22 at the distal end of the locatable guide 20. Catheter guide assemblies 11 are currently marketed and sold by Covidien LP under the name SUPERDIMENSION® Procedure Kits. Similarly, catheter guide assemblies 12 are currently sold by Covidien LP under the name EDGE™ Procedure Kits. Both kits include a handle 19, extended working channel 18, and locatable guide 20. For a more detailed description of the catheter guide assemblies including handle 19, extended working channel 18, locatable guide 20, and sensor 22, reference is made to commonly-owned U.S. patent application Ser. No. 13/836,203 filed on Mar. 15, 2013 by Ladtkow et al, the entire contents of which are hereby incorporated by reference.

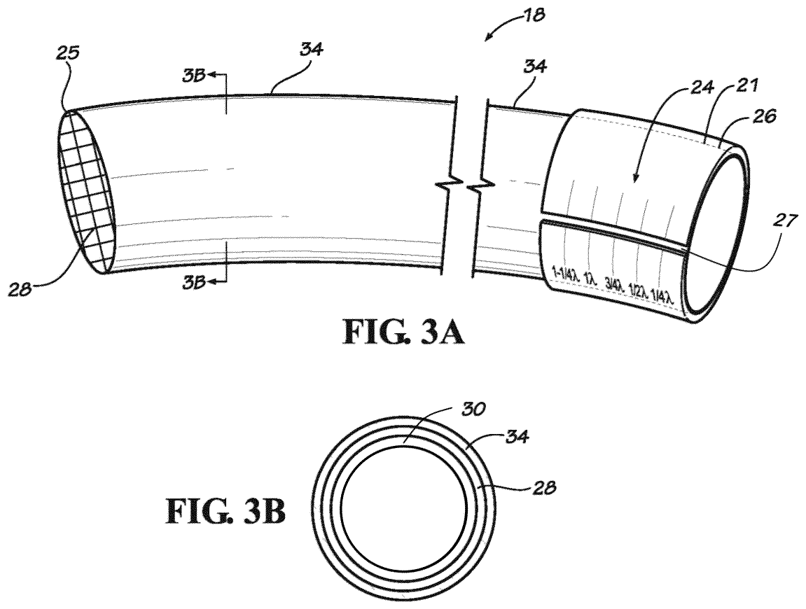

As shown in FIG. 3A, the extended working channel 18 includes a conductive inner layer 28 and an insulating layer 34. When an ablation catheter 16 is inserted into the extended working channel 18, such that the radiator 35 extends beyond the distal end 25 of the extended working channel 18, and microwave energy is applied through the coaxial cable 38 to the radiator, the conductive inner layer 28 forms an electromagnetic barrier preventing the radiation of the energy traveling down the outer conductor 32 from radiating to the tissue contacting the insulating layer 34. Essentially the conductive inner layer 28 creates a Faraday cage, significantly limiting the transmission of the energy through the extended working channel 18. The insulating layer 34 provides for additional separation of the energy from the tissue. Finally, any localized heating is effectively removed by the passage of the cooling medium along the outer conductor 32 through cooling catheter 43. The result of such an arrangement with the cooled ablation catheter 16 housed within the extended working channel 18 is that greater flexibility in the ablation catheter 16 can be achieved due to the absence of a balun, without experiencing the localized heating drawbacks which can affect ablation catheters without the emissions controlling features of a balun.

Referring to FIGS. 3A and 3B, partial views of the extended working channel 18 are shown. In one embodiment, the extended working channel is formed of two layers, a non-conductive or insulative outer layer 34 and a conductive inner layer 28. Insulating layer 34 may be formed of a medical grade flexible plastic or polymer material. The conductive inner layer 28 may be formed of a braided metallic material and substantially secured to the inner surface of the insulating layer and extends along the entire length of the inner wall of the extended working channel 18. Though depicted in FIG. 3B as concentric layers, the conductive inner layer 28 could also be imbedded in the insulative outer layer 34. The extended working channel 18 may be formed by overmolding plastic to form an outer non-conductive or insulative layer 34. The extended working channel 18 has a proximal end 21 and a distal tip 25, respectively. The extended working channel 18 is formed to receive the ablation catheter 16 (shown in FIG. 2), and, in at least one embodiment, to provide a pathway for a cooling medium to either circulate within the extended working channel 18, or to pass through the extended working channel, in both instances to cool the ablation catheter 16 when energized.

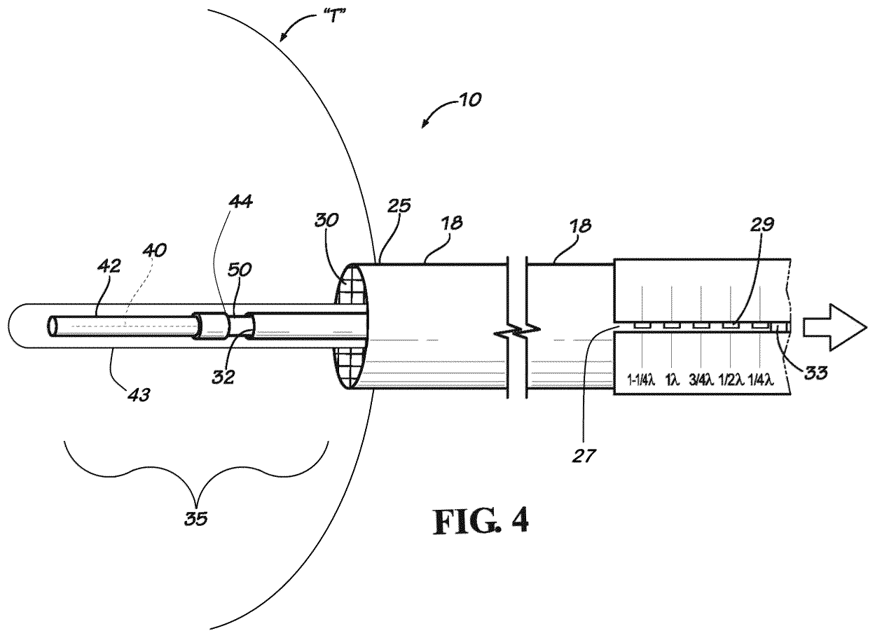

In one embodiment, a hub portion 26 is formed at the proximal end 21 of the extended working channel 18 and includes a locking mechanism 24 configured to engage the ablation catheter 16. The locking mechanism 24 may include a slot 27 that extends along the hub portion 26. The slot 27 includes a plurality of mechanical interfaces 29 (shown in FIG. 4) positioned along the opposing wall portions that define the slot 27. The mechanical interfaces 29 may be in the form of detents, protrusions, or the like that are configured to releasably engage a corresponding mechanical interface 33 (shown in FIGS. 2 and 4) provided at a proximal end of the ablation catheter 16. Engagement between the mechanical interface 33 and the plurality of mechanical interfaces 29 selectively locks the ablation catheter 16 into place within the extended working channel 18. As will be appreciated, other locking mechanisms may be employed within the scope of the disclosure.

In the embodiment of FIG. 3A, indicia are provided along the slot 27 adjacent the mechanical interfaces 29 (shown in FIG. 4) of the slot 27 and represent quarter-wavelength increments of a desired frequency of a signal transmitted from the ablation catheter 16. In embodiments, each mechanical interface 29, starting with the first mechanical interface 29, represents a quarter-wavelength value; thus, the first mechanical interface 29 represents a quarter-wavelength, the second mechanical interface represents a half-wavelength, and so on. The selection of quarter wave increments may enable the ablation catheter 16 and its use to be tuned to achieve a particularly desired ablation pattern.

Though described above with reference to an unchoked ablation catheter, the ablation catheter 16 may also incorporate a modified choke, as described in various embodiments below, without departing from the scope of the present disclosure. For example, in some embodiments, as shown in FIG. 2 a thin layer of insulating material 60 (e.g., a layer of polyethylene terephthalate (PET)) may be used to cover a portion of the outer conductor 32. This layer of insulating material 60 may assist in maintaining the braided configuration of the outer conductor 32, or may form part of a modified balun configuration.

In such a modified choke, the conductive inner layer 28 is only provided in a portion of the extended working channel 18. The electrically conductive inner layer 28 is shorted to the outer conductor 32 of the microwave ablation catheter 16 at a desired location immediately proximal a proximal end of the insulating material 60, thus creating a choke or balun by the combination of the conductive inner layer 28 and the insulating layer 60. In one example, the conductive inner layer 28 extends along an inner wall of the extended working channel 18 a distance that is approximately equal to a quarter-wavelength of a desired frequency of the signal being transmitted from the ablation catheter 16.

Upon extension of ablation catheter 16 out of the distal end of the extended working channel 18, contact is made between balun shorts 62 and the conductive inner layer 28 of the extended working channel 18, creating a balun from the combination of the extended working channel 18 and the ablation catheter 16. As will be appreciated, coordination of the location of the conductive inner surface 28 and the location of the balun shorts 62 is required to achieve the desired effect.

In a further embodiment a plurality of balun shorts 62 may be placed along the inner conductor 32 in quarter wavelength increments, resulting in a tunable microwave ablation catheter, whose position may be changed during the course of treatment to achieve a desired tissue effect. As will be appreciated, embodiments where the inner surface 28 of the extended working channel 18 is shorted to the outer conductor 32 of the ablation catheter will require electrical contacts to be placed on the catheter surrounding the ablation catheter and where necessary on the cooling catheters 43 in order to achieve the electrical short.

In an alternative embodiment of the present disclosure, an insulative layer 30 which may be formed of polytetrafluoroethylene (PTFE) may be formed on an internal surface of the conductive inner layer 28 which forms the inner wall of the extended working channel 18. In embodiments, the insulative layer 30 and/or the conductive inner layer 28 may be positioned along the interior of the extended working channel 18 so that the insulative layer 30 and the conductive inner surface 28 in combination with one or more balun shorts 62 form the balun or choke. The internal diameter of the insulative layer 30 may be such that the outer conductor 32 of the coaxial cable 38 passes through the insulative layer in sliding engagement when the ablation catheter 16 is in one of the aforementioned extended configurations. The insulative layer 30 and/or the conductive inner surface 28 and their ultimate orientation with respect to the outer conductor 32 can be adjusted during manufacture to control an overall phase, energy field profile, and/or temperature response of the ablation catheter 16.

As noted above, the ablation catheter 16 may be used in conjunction with the extended working channel 18 in several forms including cooled and uncooled. In one cooled embodiment, the ablation catheter 16 includes one or more columinal cooling catheters 43 which fully encapsulate the distal radiating portion 42. In a second cooled configuration no columinal cooling catheters 43 are used and instead the coaxial cable 38 with radiating portion 42 is simply inserted through the extended working channel 18 and cooling medium is allowed to pass through the extended working channel 18 to cool the radiating portion 42. This embodiment is an open cooling solution and the cooling medium is allowed to escape through the extended working channel 18 into the patient. In a third configuration which permits partial cooling of the coaxial cable 38 up to an insulative layer 30 which is sized to allow sliding engagement of the coaxial cable 38, the radiator 35 of the ablation catheter 16 is not cooled, and any cooling medium directed into the extended working channel 18, may not pass beyond the insulative layer 30, but the remainder of the coaxial cable 38 is cooled. The balun shorts 62, if employed, may be spaced such that they do not significantly impede the flow of the cooling medium. Cooling medium, such as CO₂ gas or de-ionized water, may also form part of the balun, providing an additional insulative layer which operates in concert with the conductive layer 28 and the balun short 62 to achieve the desired effect.

Still further, although the microwave ablation catheter 16 described here may be specific, it should be understood to those of skill in the art that other microwave ablation catheter embodiments, either simplified or more complex in structural detail, may be employed without departing from the scope of the instant disclosure.

Operation of the EMN system 10 to reach an identified target and enable the treatment of target tissue within a lung of a patient is described with reference to FIGS. 1 and 4. As an initial step, imaging of a patient “P” is typically undertaken. For example, CT images may be taken and imported into a pathway planning software. One such pathway planning software is the ILOGIC® Planning suite currently sold by Covidien LP. Typically, in such pathway planning software, the images are viewed and a clinician can identify targets within the images and generate pathways to arrive at the targets. The physician, or user, may use a prior CT scan of the patient “P” and software to construct a representation of the patient's luminal network to help determine and plan pathways to the identified target location. Once the pathway is identified and accepted, this pathway becomes a navigation plan which may be exported to navigation software such as the ILOGIC® Navigation suite currently sold by Covidien LP.

The patient “P” is then placed within the EMN system 10, as depicted in FIG. 1. The location of the patient “P”, and particularly features within the luminal structure being navigated (e.g., the lungs) are registered to the images of the navigation plan. Computer system 82, in operation with a tracking module 80, determines the position of the patient “P”, and thereby defines a set of reference coordinates, which are matched with the images of the navigation plan. As a result, the navigation software is able to superimpose the location of the sensor 22 onto the images of the navigation plan, and depict to the user the result of the manipulations of the catheter guide assembly 11, 12 on the images of the navigation plan. This system also allows the user to follow the pathway depicted in the navigation plan.

The EMN system 10 utilizes a six degrees-of-freedom electromagnetic position measuring system according to the teachings of U.S. Pat. No. 6,188,355 and published PCT Application Nos. WO 00/10456 and WO 01/67035, the entire contents of which are hereby incorporated by reference. A transmitter arrangement 76 is implemented as a board or mat positioned beneath patient “P.” A plurality of reference sensors 78 are interconnected with a tracking module 80 which derives the location of each reference sensor 78 and sensor 22 in 6 degrees of freedom. One or more of the reference sensors 78 (e.g., 3 reference sensors 78) are attached to the chest of patient “P” and their 6 degrees of freedom coordinates are sent to a computer 82 where they are used to calculate the patient coordinate frame of reference.

Once a bronchoscope 72 is inserted into the lungs of a patient “P”, the extended working channel 18 and guide catheter assembly 11, 12 including locatable guide 20 and sensor 22 are inserted into the bronchoscope 72. Bronchoscope 72 is connected to monitoring equipment 74, and typically includes a source of illumination and a video imaging system. In certain cases, the locatable guide catheter assembly 12 and extended working channel 18 may be used without a bronchoscope. After advancing the bronchoscope 72 and catheter guide assembly 11, 12, including the extended working channel 18 and the locatable guide 20, to a point of being wedged within a luminal network of the lung, the extended working channel 18 and locatable guide 20 are further advanced along the identified pathway to the target “T”. Working in conjunction with the EMN system 10, the guide catheter assembly 11, 12 is used to guide the extended working channel 18 through the luminal network of the patient “P” to the target following the planned pathway relying on the sensed location of the sensor 22 as depicted on the images in the navigation software.

Once at the identified target, the locatable guide 20 may be withdrawn and the extended working channel 18 becomes a pathway to the target “T” for subsequent diagnostic and treatment tools (e.g., biopsy tools, a guide wire, access tools, ablation catheter 16, etc.). Typically, the clinician may seek to take several biopsy samples to confirm that the target “T” is in need of treatment. In some cases, the target tissue may be directly accessed from within the lumen (such as for the treatment of the endobronchial wall for COPD, Asthma, lung cancer, etc.). However, in other instances the target is outside the luminal walls of the bronchial tree and use of the extended working channel 18 and locatable guide 20 alone do not achieve access to the target. Additional access tools may be required to pierce or sever tissue, exit the lumen, and access the target tissue (such as for the treatment of disease within the parenchyma). In embodiments, the target tissue “T” may be pierced or penetrated to allow placement of the radiator 35 of the ablation catheter 16 within the target “T” (e.g., centered within the mass for treatment). For example, a guide wire, piercing tool, a biopsy tool or the distal radiating section 42 of the ablation catheter 16 may be utilized to pierce or penetrate the target “T.”

If it is determined that the target “T” requires treatment (e.g., ablation), the ablation catheter 16 may be positioned through the bronchoscope 72 and the extended working channel 18 to enable treatment. Placement of the ablation catheter 16 may occur after the extended working channel 18 has been navigated to the target “T.” Alternatively, particularly in embodiments where sensor 22 is incorporated into the extended working channel 18 or into the ablation catheter 16 itself, the mechanical interface 33 of the ablation catheter 16 may be engaged with one of the plurality of mechanical interfaces 29 of the locking mechanism 24 and the extended working channel 18 and ablation catheter 16 may be navigated to the target “T” together. In either case, before energization of the ablation catheter 16, the radiator 35 must be extended to a position distal to the distal end 25 of the extended working channel 18 as the conductive inner surface 28, which beneficially acts as a Faraday cage, as described above, will also substantially prevent radiation from escaping the extended working channel 18 and radiating tissue.

One or more imaging modalities may be utilized to confirm that the ablation catheter 16 has been properly positioned (e.g. within the target “T”.) For example, computer tomography (CT), ultrasound, fluoroscopy, and other imaging modalities may be utilized individually or in combination with one another to confirm that the ablation catheter 16 has been properly positioned within the target “T”. One methodology employing both CT and fluoroscopy imaging modalities is described in commonly assigned U.S. application Ser. No. 12/056,123 filed on Mar. 26, 2008 by Dorian Averbruch and entitled “CT-Enhanced Fluoroscopy,” the entire contents of which are hereby incorporated by reference. Once it is confirmed that the ablation catheter 16 is properly positioned within the target tissue “T,” a user may begin with the ablation procedure and apply desired levels of microwave energy to the target “T” to achieve the desired ablation effect.

During the ablation procedure, as the temperature of the target “T” increases, the overall impedance at the tissue site may change, which may lead to an unbalanced signal between the inner and outer conductors 40 and 32. According to one embodiment, to balance the signal, a user can move ablation catheter 16 distally to disengage the mechanical interface 33 of the ablation catheter from the mechanical interface 29 on the extended working channel 18, which moves the distal radiating section 42 further from the outer conductive layer 28. If need be, the user can move mechanical interface 33 into engagement with one of the other mechanical interfaces 29 (e.g., the one that corresponds to a half-wavelength) of the extended working channel 18 to lock the distal radiating section 42 into this further position.

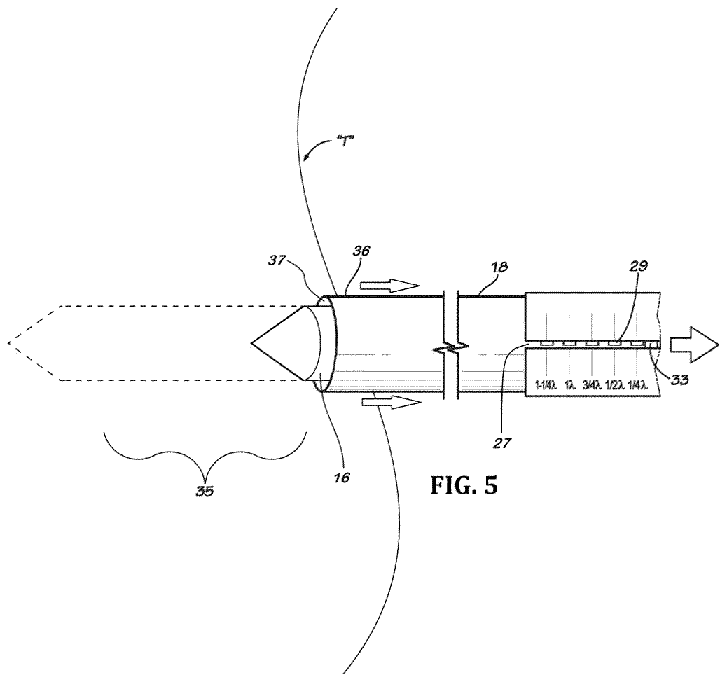

In yet another alternative embodiment of the present disclosure, the extended working channel 18 may comprise of or may be replaced by a metal hypo tube 36, as depicted in FIG. 5. In this embodiment, the metal hypo tube 36 contains a conductive inner surface 37 and may be optionally coated on its exterior surface with an insulating material as described above with respect to EWC 18. Similar to the conductive inner layer 28 (See FIG. 3A), conductive inner surface 37 functions as an electromagnetic shield for a microwave ablation catheter 16. As described above, the metal hyptotube 36 is configured to receive instruments including the locatable guide catheter 20 and sensor 22, biopsy tools and microwave ablation catheter 16, as well as others without departing from the scope of the present disclosure. During an ablation procedure, metal hypo tube 37, or extended working channel 18 including metal hypo tube 36, is inserted into a patient and positioned adjacent to, or within, target tissue “T.” The ablation catheter 16 is then placed within the metal hypo tube 36 and extended past the distal end of the metal hypo tube 36. In one embodiment, the metal hypo tube 36 may be inserted along with an ablation catheter 16 having a pointed or piercing configuration capable of piercing through tissue “T” (as depicted in FIG. 5). Although FIG. 5 depicts ablation catheter 16 with a pointed configuration, the end of the ablation catheter 16 may include any suitable configuration including but not limited to a blunt configuration, flat configuration, hemispherical configuration, bar-bell configuration, tissue piercing configuration, etc. Once the tip of the ablation catheter 16 is placed in the target tissue “T,” the metal hypo tube 36 may be retracted to expose radiator 35 of the ablation catheter 16. As explained in greater detail above, the extension of the radiator 35 out of the extended metal hypo tube enables the ablation of tissue, but the length of that extension can be varied as desired to adjust the shape and size of the ablation zone.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1-20. (canceled)

21. A microwave ablation device assembly, comprising:
a coaxial cable including:
an inner conductor;
an outer conductor separated from the inner conductor by a dielectric; and
a distal radiating section coupled to a distal portion of the inner conductor and configured to deliver microwave energy to tissue; and
a catheter including an electrically conductive inner surface electrically shorted to the outer conductor of the coaxial cable, wherein the catheter is configured such that microwave energy conducted by the outer conductor of the coaxial cable is prevented from affecting tissue in proximity to the catheter.

22. The microwave ablation device assembly according to claim 21, further comprising a location sensor disposed on at least one of the coaxial cable or the catheter.

23. The microwave ablation device assembly according to claim 22, wherein the location sensor is an electromagnetic (EM) sensor.

24. The microwave ablation device assembly according to claim 21, wherein the catheter further includes an outer insulating layer and an inner insulative layer.

25. The microwave ablation device assembly according to claim 24, wherein the electrically conductive inner surface of the catheter is disposed between at least a portion of the outer insulating layer and the inner insulative layer.

26. The microwave ablation device assembly according to claim 24, wherein the outer insulating layer is configured to separate the electrically conductive inner surface from tissue in proximity to the catheter.

27. The microwave ablation device assembly according to claim 21, wherein the electrically conductive inner surface of the catheter is braided.

28. The microwave ablation device assembly according to claim 21, wherein the catheter is configured to be received within airways of the patient for navigating the coaxial cable through the airways of the patient for placement of the distal radiating section at the target.

29. The microwave ablation device assembly according to claim 21, wherein the coaxial cable includes a layer of insulating material disposed on an outer surface of the outer conductor.

30. The microwave ablation device assembly according to claim 29, wherein the electrically conductive inner surface of the catheter is electrically shorted to the outer conductor of the coaxial cable adjacent to a proximal end of the layer of insulating material.

31. The microwave ablation device assembly according to claim 29, wherein the layer of insulating material includes polyethylene terephthalate (PET).

32. The microwave ablation device assembly according to claim 21, wherein a length of the electrically conductive inner surface of the catheter is equal to a quarter-wavelength of a frequency of the microwave energy delivered by the distal radiating section.

33. A microwave ablation device assembly, comprising:
a microwave ablation antenna configured to deliver microwave energy to tissue; and
a catheter including an electrically conductive inner surface electrically shorted to the microwave ablation antenna, wherein the catheter is configured such that microwave energy conducted by the microwave ablation antenna is prevented from affecting tissue in proximity to the catheter.

34. The microwave ablation device assembly according to claim 33, wherein the catheter further includes an outer insulating layer and an inner insulative layer.

35. The microwave ablation device assembly according to claim 34, wherein the electrically conductive inner surface of the catheter is disposed between at least a portion of the outer insulating layer and the inner insulative layer.

36. The microwave ablation device assembly according to claim 34, wherein the outer insulating layer is configured to separate the electrically conductive inner surface from tissue in proximity to the catheter.

37. The microwave ablation device assembly according to claim 33, wherein the electrically conductive inner surface of the catheter is braided.

38. The microwave ablation device assembly according to claim 33, wherein the catheter is configured to be received within airways of the patient for navigating the microwave ablation antenna through the airways of the patient for placement of the microwave ablation antenna at the target.

39. The microwave ablation device assembly according to claim 33, wherein a length of the electrically conductive inner surface of the catheter is equal to a quarter-wavelength of a frequency of the microwave energy delivered by the microwave ablation antenna.

40. A microwave ablation device assembly, comprising:
a microwave ablation antenna configured to deliver microwave energy to tissue, the microwave ablation antenna including an outer conductor and an inner conductor disposed coaxially within the outer conductor; and
a catheter including an electrically conductive inner surface electrically shorted to the outer conductor of the microwave ablation antenna.
